(19) United States
(12) Patent Application Publication
(10) Pub. No.: US 2026/0004921 A1
(43) Pub. Date: Jan. 1, 2026

(54) COMPUTER-IMPLEMENTED SYSTEM AND METHOD FOR REMOTE MONITORING AND EARLY DETECTION OF HEALTH RISKS IN AN INDIVIDUAL, PARTICULARLY AN ELDERLY PERSON

(71) Applicant: PRESAGE, PARIS (FR)

(72) Inventor: JACQUES-HENRI VEYRON, NEUILLY-SUR-SEINE (FR)

(21) Appl. No.: 19/313,857

(22) Filed: Aug. 28, 2025

Related U.S. Application Data
(63) Continuation-in-part of application No. 17/298,915, filed on Jun. 1, 2021, now abandoned.

(30) Foreign Application Priority Data
Nov. 29, 2018 (FR) 1872055

(51) Int. Cl. G16H 40/20; G16H 10/60; G16H 15/00; G16H 40/67; G16H 50/30; G16H 80/00

(52) U.S. Cl. CPC G16H 40/20; G16H 10/60; G16H 15/00; G16H 40/67; G16H 50/30; G16H 80/00

(57) ABSTRACT

A computer-implemented method and system for assessing, within a predetermined future time widow, a risk of a hospital transfer of a monitored individual. In the data collection phase, an outcome data relating to hospital admission and a plurality of dated status sheets are acquired having at least four indicators. In the model training phase, a training dataset is built, and a model of machine learning algorithm is trained on the training dataset. In the operational analysis phase, new data sheets at distinct time points are acquired, the trained model is applied to compute a risk score of the hospital transfer, at predefined analysis intervals, the risk score is updated and an alert is automatically generated when the risk score or its temporal trend exceeds the threshold and is transmitted to a monitoring platform or a healthcare professional.

### RELATED APPLICATIONS

This application is a continuation-in-part application of application Ser. No. 17/298,915 filed Jun. 1, 2021, which is a § 371 application from PCT/EP2019/081658 filed Nov. 18, 2019, which claims priority from French Patent Application No. 18 72055 filed Nov. 29, 2018, each of which is herein incorporated by reference in its entirety.

### TECHNICAL FIELD OF THE INVENTION

The invention lies in the field of computer science and, more particularly, in the field of computer-implemented systems and methods for health monitoring.

More specifically, the invention relates to a data processing system and method configured to determine the risk that an individual will require transfer to an emergency department or other healthcare facility.

The invention finds particular application in the remote monitoring of individuals living autonomously at home, generally outside of a medicalized environment. Such individuals are typically elderly persons and often present multiple chronic pathologies. The system of the invention makes it possible to monitor these persons continuously and to generate early alerts, thereby facilitating timely preventive intervention by caregivers or healthcare professionals.

### BACKGROUND OF THE INVENTION

Techniques for remote monitoring of the condition of an individual are known from the prior art.

In general, such techniques are based on the use of sensors measuring at least one physiological data of the individual, amongst the heart rate, the blood pressure, the temperature, the oxygen or glucose level in the blood, etc.

In particular, continuous monitoring techniques relying on connected objects such as smartwatches or similar wearable devices have been proposed. While these devices can capture physiological signals, they present significant drawbacks for older or vulnerable individuals. First, patient adherence is problematic, since proper configuration and continuous wearing of the device may not always be possible, especially in the presence of cognitive decline or patient refusal. Second, such devices require regular recharging-typically during the night-which prevents monitoring precisely at times when nocturnal health events may occur. Third, these wearable devices operate essentially as reactive monitoring tools, detecting changes after they have already manifested, rather than providing preventive prediction of adverse events.

The major drawback of these techniques is that they require a regular recording of these physiological data for the processing of the data to be reliable to determine the condition of the individual.

This regular recording may further turn out to be very binding for the individual and even requiring a regular intervention of a medical attendant to perform more technical acts, such as a blood sample analyzed subsequently.

Moreover, the monitoring of a population at risk turns out to be tedious for healthcare professionals who have to analyze the physiological data of a large number of individuals.

To facilitate the work of healthcare professionals, automatic data processing techniques have been suggested, based in particular on statistical analyses of physiological data on a large scale.

In general, such techniques are dedicated to the prediction of a particular pathology and consequently turn out to be unreliable to determine a risk of admission of an individual having a multitude of pathologies at once to the emergency department.

None of the current systems does allow addressing all of the required needs simultaneously, namely providing a technique for determining, more efficiently, a risk of transfer of an individual having several pathologies at once to the emergency department, in the near future within seven days, that is reliable and merely intrusive for the individual.

### Object and Summary of the Invention

These objectives, as well as other ones that will come out only later on, are achieved using a computer-implemented method executed by a processor for assessing, within a predetermined future time window, a risk of hospital transfer of a monitored individual, the method comprising:
a data collection phase, comprising steps of:
acquiring, for a group of individuals, a plurality of dated status sheets, each status sheet comprising at least four binary observational indicators relating to health, social interaction, behavior, and/or physical or sensory capabilities of the individual, without any physiological parameter;
acquiring outcome data relating to hospital admission events for at least some individuals of the group;
a model training phase, comprising steps of:
building an initial training set combining:
the acquired dated status sheets,
the outcome data, and
a set of “negative” status sheets from individuals without imminent transfer; training, by the processor, a model of a machine learning algorithm on the training set;
an operational analysis phase, comprising steps of:
acquiring, for the monitored individual, a plurality of new status sheets established at distinct time points;
applying the trained model to compute a numerical risk score of hospital transfer;
updating the risk score at predefined analysis intervals based on at least a new status sheet;
automatically generating an alert when the risk score or its temporal trend exceeds a threshold, and transmitting the alert to a monitoring platform or healthcare professional.

In particular embodiments of the invention, the data collection phase comprises also:
generating, by the processor, an augmented training dataset by applying to certain status sheets at least the following data augmentation transformations:
controlled random modification of at least one indicator value according to historically derived patterns; and addition of derived variables representing the temporal evolution of an indicator compared to the individual's previous status sheets;

In particular embodiments of the invention, the binary observational indicators are selected among:
indicators related to the health condition of the individual, including:
   A1. the individual has swollen legs;
   A2. the individual has difficulties in breathing;
   A3. the individual is feverish;
   A4. the individual has pains;
relational-type indicators, including:
   B1. the individual is indifferent;
   B2. the individual is not very communicative;
   B3. the individual lives alone since at least seven days;
   B4. the individual has contacts or visits with his entourage;
behavioral-type indicators, including:
   C1. the individual refuses help with toileting;
   C2. the individual does not recognize the companion;
   C3. the individual forgets when the companion has come by;
   C4. the individual communicates inconsistently;
   C5. the individual is aggressive;
   C6. the individual is sad;
   C7. the individual stores objects in inappropriate locations;
   C8. the individual seems tired;
   C9. the individual refuses the intervention of the companion;
indicators representative of the physical and sensory capabilities of the individual, including:
   D1. the individual stands up;
   D2. the individual moves at his home;
   D3. the individual performs personal hygiene;
   D4. the individual prepares his meals;
   D5. the individual leaves his home;
   D6. the individual eats;
   D7. the individual falls.

In particular embodiments of the invention, at least one binary indicators of the status sheets is populated by an automatic analysis of data provided by a sensor.

In particular embodiments of the invention, geolocated and dated epidemiological information relating to the outside temperature, relating to influenza-like and/or acute diarrhea illnesses are collected during the data collection phase in order to be included in the training set of the model.

In particular embodiments of the invention, at least nine binary observational indicators are included in each status sheet, the nine indicators comprising: A2, A3, A4, B2, B4, C6, D2, D4, and D7.

In particular embodiments of the invention, the nine binary indicators further comprise C7, thereby forming a list of ten binary indicators.

In particular embodiments of the invention, each binary indicator is associated with a sub-indicator representative of a temporal evolution of the corresponding indicator, the sub-indicator being selected among “improvement,” “stabilization,” or “degradation.”

In particular embodiments of the invention, at least one of the binary indicators is automatically populated using data from a motion detection sensor selected from a presence sensor, a camera, or an infrared camera.

In particular embodiments of the invention, the motion detection sensor further comprises a facial recognition algorithm configured to distinguish between multiple individuals within the monitored environment.

In particular embodiments of the invention, at least one of the binary indicators is automatically populated using data from a RFID or NFC sensor cooperating with a tag secured to a monitored object.

In particular embodiments of the invention, the monitored object is selected from a pair of slippers, spectacles, a dental appliance, a hearing aid, a phone, or a remote control.

In particular embodiments of the invention, at least one of the binary indicators is automatically populated using a weight sensor configured to detect unusual variations in the individual's body weight.

In particular embodiments of the invention, the alert generated comprises at least one of: a text message, an email, or a push notification transmitted through a secure communication channel.

In particular embodiments of the invention, the alert includes an identification of the monitored individual, the computed risk score, and at least one binary indicator contributing to the elevated risk.

In particular embodiments of the invention, the machine learning algorithm is selected from a Random Forest classifier, a Gradient Boosted Trees model, a Support Vector Machine, or a shallow neural network.

In particular embodiments of the invention, the parameters of the machine learning algorithm are periodically updated by recording newly acquired status sheets and dates of hospital transfer for the monitored individual.

In particular embodiments of the invention, the training of the machine learning algorithm comprises generating, for each individual of the group, a temporal feature vector derived from a plurality of dated status sheets, each temporal feature vector encoding changes in binary indicators over at least two consecutive status sheets.

In particular embodiments of the invention, the temporal feature vector further comprises trend attributes representing smoothed variations of each indicator over a sliding analysis window.

In particular embodiments of the invention, the machine learning algorithm is configured to assign, during training, weighting coefficients to each indicator based on statistical correlation of the indicator's temporal evolution with recorded hospital transfers.

In particular embodiments of the invention, applying the trained model to a new status sheet comprises:
generating a temporal difference vector between the new status sheet and at least one previously recorded status sheet of the monitored individual;
combining the temporal difference vector with the stored parameters of the model; and
computing a numerical risk score based on the combination.

In particular embodiments of the invention, the temporal difference vector includes categorical values indicating persistence, improvement, or deterioration of at least one binary indicator.

In particular embodiments of the invention, updating the risk score at predefined intervals comprises dynamically recalculating the temporal feature vectors as new status sheets are added, and re-applying the trained parameters without re-training the entire model.

In particular embodiments of the invention, retraining of the model comprises an incremental learning step, the incremental learning step including:
recording a new outcome event corresponding to hospital transfer of the monitored individual,
appending the corresponding status sheets to the training dataset, and
updating the parameters of the machine learning algorithm based on both the new and previously stored datasets.

In a second aspect, the invention relates to a computer-implemented method executed by a processor for assessing, within a predetermined future time window, a risk of hospital transfer of a monitored individual, the method comprising:
a data collection phase, comprising steps of:
acquiring, for a group of individuals, a plurality of dated status sheets, each status sheet comprising:
a plurality of binary observational indicators relating to health, social interaction, behavior, and/or physical or sensory capabilities of the individual;
at least one physiological parameter selected from blood pressure, heart rate, body temperature, oxygen saturation, or body weight;
acquiring outcome data relating to hospital admission events for at least some individuals of the group;
a model training phase, comprising steps of:
building a training set combining:
the acquired dated status sheets,
the outcome data, and
a set of “negative” status sheets from individuals without imminent transfer;
training, by the processor, a model of a machine learning algorithm comprising at least a classifier that integrates observational indicators as primary features and physiological parameters as secondary features, on the training set;
an operational analysis phase, comprising steps of:
acquiring, for the monitored individual, a plurality of new status sheets established at distinct time points;
extracting, for each new status sheet, temporal attributes representing changes in observational indicators as well as smoothed trends of physiological parameters;
applying the trained hybrid model to compute a numerical risk score, wherein the contribution of observational indicators is weighted higher than that of physiological parameters;
updating the risk score at predefined analysis intervals based on at least a new status sheet;
automatically generating an alert when the risk score or its temporal trend exceeds a threshold, and transmitting the alert to a monitoring platform or healthcare professional.

In particular embodiments of the invention, the binary observational indicators are selected among:
indicators related to the health condition of the individual, including:
   A1. the individual has swollen legs;
   A2. the individual has difficulties in breathing;
   A3. the individual is feverish;
   A4. the individual has pains;
relational-type indicators, including:
   B1. the individual is indifferent;
   B2. the individual is not very communicative;
   B3. the individual lives alone since at least seven days;
   B4. the individual has contacts or visits with his entourage;
behavioral-type indicators, including:
   C1. the individual refuses help with toileting;
   C2. the individual does not recognize the companion;
   C3. the individual forgets when the companion has come by;
   C4. the individual communicates inconsistently;
   C5. the individual is aggressive;
   C6. the individual is sad;
   C7. the individual stores objects in inappropriate locations;
   C8. the individual seems tired;
   C9. the individual refuses the intervention of the companion;
   C10. the individual gets dressed;
indicators representative of the physical and sensory capabilities of the individual, including:
   D1. the individual stands up;
   D2. the individual moves at his home;
   D3. the individual performs personal hygiene;
   D4. the individual prepares his meals;
   D5. the individual leaves his home;
   D6. the individual eats;
   D7. the individual falls.

In particular embodiments of the invention, the hybrid model assigns a weighting coefficient to each observational indicator that is greater than the weighting coefficient assigned to each physiological parameter.

In particular embodiments of the invention, the hybrid model comprises two classifiers executed in parallel, a first classifier trained primarily on binary observational indicators and a second classifier trained on physiological parameters, and wherein the outputs of the classifiers are combined through weighted voting to generate the risk score.

In a third aspect, the invention relates to a computer-implemented method executed by a processor for predicting, within a predetermined future time window, an onset of at least one symptom in a monitored individual in an everyday environment, the method comprising:
a data collection phase, comprising steps of:
acquiring, for a group of individuals, a plurality of dated status sheets, each status sheet comprising at least four binary observational indicators relating to health, social interaction, behavior, and/or physical or sensory capabilities of the individual, without any physiological parameter;
acquiring outcome data relating to occurrences of symptoms, such as falls, malnutrition, depression, or swollen legs, for at least some individuals of the group;
a model training phase, comprising steps of:
building an initial training set combining:
the acquired dated status sheets,
the outcome data, and
a set of “negative” status sheets from individuals without imminent transfer; training, by the processor, a model of a machine learning algorithm on the training set;
an operational analysis phase, comprising steps of:
acquiring, for the monitored individual, a plurality of new status sheets established at distinct time points;
applying the trained model to compute a numerical risk score for the at least one symptom, the symptom not having been previously observed for the monitored individual in the recorded status sheets;
updating the risk score at predefined analysis intervals based on at least a new status sheet;
automatically generating an alert when the risk score or its temporal trend exceeds a threshold, and transmitting the alert to a monitoring platform or healthcare professional.

In particular embodiments of the invention, the binary observational indicators are selected among:
indicators related to the health condition of the individual, including:
   A1. the individual has swollen legs;
   A2. the individual has difficulties in breathing;
   A3. the individual is feverish;
   A4. the individual has pains;
relational-type indicators, including:
   B1. the individual is indifferent;
   B2. the individual is not very communicative;
   B3. the individual lives alone since at least seven days;
   B4. the individual has contacts or visits with his entourage;
behavioral-type indicators, including:
   C1. the individual refuses help with toileting;
   C2. the individual does not recognize the companion;
   C3. the individual forgets when the companion has come by;
   C4. the individual communicates inconsistently;
   C5. the individual is aggressive;
   C6. the individual is sad;
   C7. the individual stores objects in inappropriate locations;
   C8. the individual seems tired;
   C9. the individual refuses the intervention of the companion;
indicators representative of the physical and sensory capabilities of the individual, including:
   D1. the individual stands up;
   D2. the individual moves at his home;
   D3. the individual performs personal hygiene;
   D4. the individual prepares his meals;
   D5. the individual leaves his home;
   D6. the individual eats;
   D7. the individual falls.

In particular embodiments of the invention, the symptom predicted is selected from a risk of falling, a risk of malnutrition, a risk of depression, or a risk of swollen legs.

In particular embodiments of the invention, the risk score for the symptom is generated only when the symptom has not been previously observed for the monitored individual.

In particular embodiments of the invention, the trained model produces an explainability output comprising at least one feature-importance score for the indicators that contributed to the prediction of the symptom.

In a fourth aspect, the invention relates to a computer system for remote monitoring and assessing, within a predetermined future time window, a health risk in a monitored individual, the system comprising:
a processor; and
a memory storing instructions which, when executed by the processor, cause the processor to:
acquire, for a group of individuals, a plurality of dated status sheets, each status sheet comprising at least four binary observational indicators relating to health, social interaction, behavior, and/or physical or sensory capabilities of the individual, without any physiological parameter;
acquire outcome data relating to hospital admission events for at least some individuals of the group;
build an initial training set combining the acquired status sheets, the outcome data, and a set of “negative” status sheets;
train a model of a machine learning algorithm on the training set;
acquire a plurality of new status sheets of the monitored individual established at distinct time points;
apply the trained model to compute a numerical risk score of hospital transfer or an onset of at least one symptom in the monitored individual;
update the risk score at predefined analysis intervals; and
automatically generate and transmit an alert when the risk score or its temporal trend exceeds a threshold.

In particular embodiments of the invention, the memory further stores instructions for generating a temporal feature vector encoding changes of binary indicators across at least two consecutive status sheets.

In particular embodiments of the invention, the processor is further configured to associate each binary indicator with a sub-indicator selected from “improvement,” “stabilization,” or “degradation.”

In particular embodiments of the invention, the computer system further comprises at least one motion detection sensor selected from a presence sensor, a camera, or an infrared camera, the sensor being configured to automatically populate at least one binary indicator of the status sheet.

In particular embodiments of the invention, the processor is further configured to update the parameters of the machine learning algorithm incrementally by appending newly acquired status sheets and recorded outcome events to the training dataset.

In particular embodiments of the invention, the alert comprises a text message, an email, or a push notification transmitted through a secure communication channel to a healthcare professional.

Finally, the invention relates also to a non-transitory computer-readable medium storing instructions that, when executed by a processor, cause the processor to perform the method of any one of the previous embodiments.

In others words, the objectives of the invention, as well as other ones that will come out only later on, are achieved using a data processing system for determining a risk factor of an imminent transfer of an individual to the emergency department.

The objective of the present invention is to enable predicting a transfer of the individual to the emergency department within the next seven days with a predictive performance higher than 50%, preferably at least 65%, more preferably higher than 70%.

In general, such a system comprises a computer server provided with a microprocessor and a computer memory.

According to the invention, the data processing system also comprises:
a database storing a plurality of status sheets for each person of a group of persons and a database storing the dates of transfers of said group of persons to the emergency department, each status sheet being dated and including a list of monitoring indicators, each indicator having a value selected in a list of two predetermined values according to the status of the corresponding person;
means for generating the parameters of a machine learning algorithm from the status sheets and the dates of transfers of the group of persons to the emergency department;
means for filling a status sheet of the individual, the status sheet including the list of monitoring indicators, each indicator having a value selected in a list of two predetermined values according to the status of the individual;
means for determining the risk factor by analysis of a plurality of status sheets of the individual thanks to the machine learning algorithm whose parameters have been generated beforehand, the status sheets of the individual being established at distinct time points;
means for generating a warning when the risk factor exceeds a predetermined threshold.

Thus, it is possible to predict a risk of transfer to the emergency department within an imminent time period, generally within the next seven days.

It should be highlighted that the determination of the risk factor is performed without any analysis of the physiological data of the individual, these not being included in the status sheet. Moreover, the value of the risk factor does not provide any indication with regards to a pathology of the individual.

In general, the status sheet comprises a plurality of monitoring indicators whose values may be determined by the individual, by a caregiver or by a companion, without requiring any prior medical knowledge. In general, the possible values consist of a positive value (for example: “Yes”) and a negative value (for example: “No”). In other words, these monitoring indicators are determined according to an observation of the individual.

Thus, the monitoring of the condition of the person is simple to implement and merely intrusive.

Furthermore, the parameters generated from a very large number of status sheets and of transfers to the emergency department recorded beforehand allow determining the risk factor for the individual by analyzing the evolution of the status sheets recorded on a regular basis, for example every week or two to three times a week.

It should be highlighted that the monitored individual is generally an elderly person rarely having only one pathology but several pathologies at once, which increases the risk factor of transfer to the emergency department.

By using indicators having a limited number of possible values, it is thus possible to deduce, thanks to a large-scale analysis, a risk factor of transfer of the individual to the emergency department.

The warning may be in the form of text such as a message intended for a practitioner, in the form of light and/or in the form of sound, who, consequently, can monitor the condition of the individual without having to make regular trips. For example, the predetermined threshold may be in the range of 40%, 50% or 60%.

A risk indicator may also be determined according to the risk factor to indicate whether the risk is considerable, whether vigilance is needed or whether the risk is low.

It should also be highlighted that the invention is implemented by a computer allowing processing a very large number of data, in general greater than a few tens of data, in a short period of time. This automatic processing allows determining the parameters of the machine learning algorithm that will be used for the determination of the risk factor of a transfer to the emergency department in the near future.

Advantageously, the monitoring indicators of the list of each status sheet are selected among:
indicators related to the health condition of the individual, such as:
   A1. the individual has swollen legs;
   A2. the individual has difficulties in breathing;
   A3. the individual is feverish;
   A4. the individual has pains;
relational-type indicators, such as:
   B1. the individual is indifferent;
   B2. the individual is not very communicative;
   B3. the individual lives alone since at least seven days;
   B4. the individual has contacts or visits with his entourage;
behavioral-type indicators, such as:
   C1. the individual refuses help with toileting;
   C2. the individual does not recognize the companion;
   C3. the individual forgets when the companion has come by;
   C4. the individual communicates inconsistently;
   C5. the individual is aggressive;
   C6. the individual is sad;
   C7. the individual stores objects in inappropriate locations;
   C8. the individual seems tired;
   C9. the individual refuses the intervention of the companion;
indicators representative of the physical and sensory capabilities of the individual, such as:
   D1. the individual stands up;
   D2. the individual moves at his home;
   D3. the individual performs personal hygiene;
   D4. the individual prepares his meals;
   D5. the individual leaves his home;
   D6. the individual eats;
   D7. the individual falls.

These indicators may be accompanied with indicators relating to the caregiver such as:
E1. the caregiver is sad;
E2. the caregiver is exhausted.

Advantageously, all or part of the monitoring indicators are associated to a sub-indicator indicating the evolution of said indictor in comparison with the last filling of the status sheet. The sub-indicator is selected amongst three values generally corresponding to an improvement of the status, to a stabilization of the status and to a degradation of the status.

Preferably, the list of the monitoring indicators of each status sheet comprises at least four monitoring indicators.

In other words, the data analysis for determining the risk factor of a transfer to the emergency department is performed on at least four monitoring indicators.

More preferably, the list of the monitoring indicators of each status sheet comprises at least nine monitoring indicators.

Advantageously, the list of the monitoring indicators of each status list is identical.

Preferably, the list of the monitoring indicators of each status sheet comprises all or part of the following nine monitoring indicators:
A2. the individual has difficulties in breathing;
A3. the individual is feverish;
A4. the individual has pains;
B2. the individual is not very communicative;
B4. the individual has contacts or visits with his entourage;
C6. the individual is sad;
D2. the individual moves at his home;
D4. the individual prepares his meals;
D7. the individual falls.

Advantageously, the list of the monitoring indicators of each status sheet comprises at least ten monitoring indicators including:
A2. the individual has difficulties in breathing;
A3. the individual is feverish;
A4. the individual has pains;
B2. the individual is not very communicative;
B4. the individual has contacts or visits with his entourage;
C6. the individual is sad;
C7. the individual stores objects in inappropriate locations;
D2. the individual moves at his home;
D4. the individual prepares his meals;
D7. the individual falls.

In particular embodiments of the invention, the data processing system also comprises a device for filling a status sheet.

The device for filling a status sheet may be a portable computer terminal provided with means for communication with the computer server, such as a smartphone or a tablet.

It should be highlighted that, in general, the computer server is not at the home of the individual but located in a remote location. The communication being generally performed via the Internet network and/or the mobile telecommunication network.

In particular embodiments of the invention, the data processing system also comprises at least one sensor transmitting data to a collection terminal configured to process the data and to communicate with the computer server.

To this end, the sensor generally comprises Bluetooth or Wi-Fi type wireless communication means in order to transmit the acquired data.

In general, the collection terminal comprises a microprocessor, a computer memory in order to store the transmitted data and means for communication with the computer server.

Advantageously, the sensor is a sensor for detecting movements.

Such a sensor may be a presence sensor, a camera or an infrared camera.

Thus, depending on the positioning of the sensor(s), all or part of the indicators D1 to D7 could be automatically determined.

In the case where the movement detection sensor is a camera or an infrared camera, a processing of the images is generally performed by the collection terminal.

Advantageously, the sensor is a RFID-type (acronym of “Radio Frequency Identification”) sensor or an NFC-type (acronym of “Near-Field Communication”) sensor cooperating with a RFID or NFC tag secured to an object.

As soon as one of the monitored objects is detected as being stored at an unusual location, the indicator C7 automatically takes on the positive value.

In general, the monitored object is an object that is commonly used by the individual such as a pair of slippers, a pair of spectacles, a dental appliance, a hearing aid, a phone or a remote-control.

In particular embodiments of the invention, the sensor is a weight sensor.

Thus, it is possible to estimate the evolution of the weight of the person by detecting an unusual weight.

In particular embodiments of the invention, the data processing system also comprises a database storing the geolocated and dated epidemiological information relating to the temperature of the commune, relating to influenza-like and/or acute diarrhea illnesses.

Thus, it is possible to improve the generation of the parameters of the machine learning algorithm.

According to a second aspect, the invention relates to a data processing method for the prediction of a risk factor of an imminent transfer of an individual to the emergency department.

Such a method comprises a learning phase and an analysis phase.

The learning phase comprises steps of:
acquisition of a plurality of status sheets for each person of a group of persons, each status sheet being dated and including a plurality of monitoring indicators of the corresponding person, each indicator having a value selected in a list of two predetermined values;
acquisition of the dates of transfers of said group of persons to the emergency department;
analysis of the status sheets and of the dates of transfers of all or part of the persons of the group to the emergency department;
generation of the parameters of a machine learning algorithm from the previous analysis.

The analysis phase comprises steps of:
acquisition of a plurality of status sheets of the individual at distinct time points, each sheet comprising a plurality of monitoring indicators of the individual, each indicator having a value selected in a list of two predetermined values;
determination of a value representative of a risk of an imminent transfer of the individual to the emergency department in the coming days, called risk factor, from the analysis of the evolution of the status sheets of the individual over a predetermined period by the machine learning algorithm whose parameters have been generated during the learning phase;
generation of a warning when the risk factor exceeds a predetermined threshold.

In particular implementations of the invention, the analysis step of the learning phase also takes into account the geolocated epidemiological information.

In particular implementations of the invention, the data processing method also comprises a step of recording the status sheets and the date of transfer of the individual to the emergency department and of updating the parameters of the machine learning algorithm.

The invention also relates to a computer program product implementing the data processing method according to any one of the preceding implementation modes.

### BRIEF DESCRIPTION OF THE FIGURES

Other advantages, objects and particular features of the present invention will come out from the following non-limiting description of at least one particular embodiment of the devices object of the present invention, with reference to the appended drawings, wherein:

FIG. 1 is a simplified diagram of an example of processing system according to the invention;

FIG. 2 is a flowchart of an example of processing method implemented by the processing system of FIG. 1;

FIGS. 3A-3F are six graphs showing an example of comparison of the predictive results obtained by the method of FIG. 2 according to different combinations of indicators.

FIG. 4 is a simplified diagram of another example of processing system according to the invention;

FIGS. 5A-5D comprise four radar charts illustrating the relative weight of each indicator in predicting respectively a risk of falling in FIG. 5A, a risk of malnutrition in FIG. 5B, a risk of depression in FIG. 5C and a risk of swollen legs in FIG. 5D.

FIG. 6 is a flowchart of an example of processing method implemented by the processing system of FIG. 4;

FIGS. 7A-7d comprise six graphs showing an example of comparison of the predictive results obtained by the method of FIG. 6 according to different combinations of indicators.

FIG. 8 illustrates, in the form of a flowchart, a generic embodiment of the data processing method implemented by the system of FIG. 1 or FIG. 4.

### DETAILED DESCRIPTION OF EMBODIMENTS OF THE INVENTION

The present description is provided in a non-limiting way, each feature of one embodiment may be advantageously combined with any other feature of any other embodiment.

As of now, it should be noted that the figures are not to the scale.

### EXAMPLE OF A PARTICULAR EMBODIMENT OF THE INVENTION

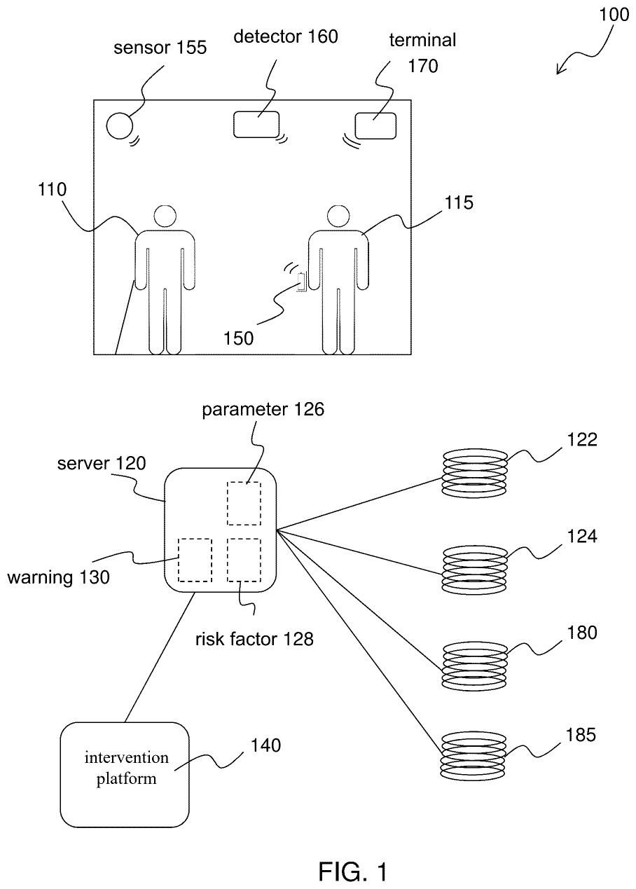

FIG. 1 is a simplified diagram of a data processing system 100 for the determination of a risk factor of an imminent transfer of an individual 110 to the emergency department.

The data processing system 100 comprises a computer server 120 provided with a microprocessor and with a computer memory in which is stored a machine learning algorithm allowing determining a value representative of the risk of the individual 110 being admitted to the emergency department in the near future, corresponding in general to the next seven days. Later on, this value is called risk factor.

It should be highlighted that the machine learning algorithm is generally selected amongst machine learning techniques, such as a “random forest” type algorithm.

In particular, the determination of the risk factor is performed in a tricky and surprising way by analyzing the evolution of status sheets of the individual 110, each status sheet being established at distinct time points and including a plurality of monitoring indicators of the individual, each indicator having a value selected in a list of two predetermined values, generally a positive value (“Yes”) and a negative value (“No”).

It should be highlighted that the status sheets being in particular devoid of any physiological data of the individual 110, they can be filled by everyone. Thus, for example, each status sheet can be filled by a caregiver 115 vising the individual 110. It should be highlighted that a companion of the individual 110 can fill the status sheet instead of the caregiver 115.

Each status sheet comprises a list of monitoring indicators generally selected among the following overall list of monitoring indicators:
indicators related to the health condition of the individual, such as:
   A1. the individual has swollen legs;
   A2. the individual has difficulties in breathing;
   A3. the individual is feverish;
   A4. the individual has pains;
relational-type indicators, such as:
   B1. the individual is indifferent;
   B2. the individual is not very communicative;
   B3. the individual lives alone since at least seven days;
   B4. the individual has contacts or visits with his entourage;
behavioral-type indicators, such as:
   C1. the individual refuses help with toileting;
   C2. the individual does not recognize the companion;
   C3. the individual forgets when the companion has come by;
   C4. the individual communicates inconsistently;
   C5. the individual is aggressive;
   C6. the individual is sad;
   C7. the individual stores objects in inappropriate locations;
   C8. the individual seems tired;
   C9. the individual refuses the intervention of the companion;
indicators representative of the physical and sensory capabilities of the individual, such as:
   D1. the individual stands up;
   D2. the individual moves at his home;
   D3. the individual performs personal hygiene;
   D4. the individual prepares his meals;
   D5. the individual leaves his home;
   D6. the individual eats;
   D7. the individual falls.

These indicators may be accompanied with indicators relating to the caregiver such as:
E1. the caregiver is sad;
E2. the caregiver is exhausted.

It should be highlighted that each indicator is representative of a status and that an equivalent formulation of one or several indicator(s) could be used without any notable alteration of the obtained results.

Quite advantageously, the status sheet comprises all or part of the list of the following nine monitoring indicators:
A2. the individual has difficulties in breathing;
A3. the individual is feverish;
A4. the individual has pains;
B2. the individual is not very communicative;
B4. the individual has contacts or visits with his entourage;
C6. the individual is sad;
D2. the individual moves at his home;
D4. the individual prepares his meals;
D7. the individual falls.

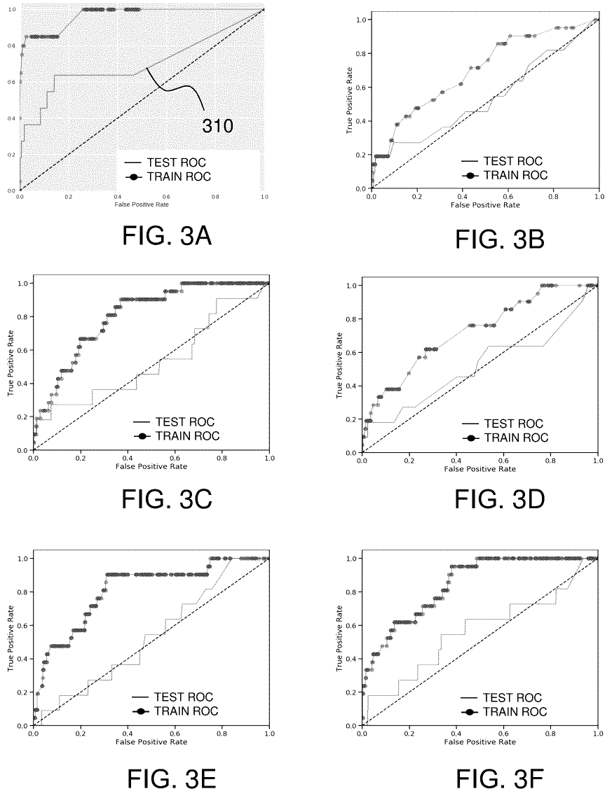

By analyzing the joint evolution of these nine monitoring indicators, it is, quite surprisingly, possible to predict a transfer of the individual 110 to the emergency department in the next seven days with a prediction rate in the range of 70%, which allows obtaining a very rapid support of the individual 110 thereby avoiding his condition getting worse. The predictive performance of this combination of nine indicators is illustrated in FIG. 3A described in more details later on. It should be highlighted that the prediction rate of a transfer to the emergency department in the next fourteen days when taking into account these nine monitoring indicators is in the range of 63%.

In variants of this particular embodiment of the invention, the status sheet comprises the list of the following ten monitoring indicators:
A2. the individual has difficulties in breathing;
A3. the individual is feverish;
A4. the individual has pains;
B2. the individual is not very communicative;
B4. the individual has contacts or visits with his entourage;
C6. the individual is sad;
C7. the individual stores objects in inappropriate locations;
D2. the individual moves at his home;
D4. the individual prepares his meals;
D7. the individual falls.

In these variants, the indicator C7 has been added with regards to the list comprising nine indicators, which allows improving the prediction of the risk of transfer to the emergency department.

It should be highlighted that the lists the nine or ten monitoring indicators constitute non-limiting examples of the invention and other combinations of at least nine indicators amongst the overall list of monitoring indicators could allow obtaining similar prediction results.

Moreover, in the case where the status sheet only the following four indicators are filled:
A3. the individual is feverish;
B4. the individual has contacts or visits with his entourage;
D2. the individual moves at his home; and
D4. the individual prepares his meals, the predictive performance of a transfer to the emergency department is in the range of 55%. The predictive performance is similar when the indicator A4 “the individual has pains” is added to this list of four indicators. The predictive performance of this combination of five indicators is illustrated in FIG. 3B.

All or part of the monitoring indicators of the status sheet may be associated to a sub-indicator indicating a precision related to said monitoring indicator, namely an evolution of the status object of said indictor in comparison with the last filling of the status sheet. The sub-indicator is selected amongst three values generally corresponding to an improvement of the status (for example: “better”), to a stabilization of the status (for example: “same”) and to a degradation of the status (for example: “less well”). This sub-indicator allows adding another dimension regarding the indicator whose value has not been modified between two successively filled status sheets.

In general, a sub-indicator is associated to the monitoring indicators A1 to A4, B1, C8, D2, D6 and/or D7.

Thanks to the use of the sub-indicators, it is also possible to improve the prediction of the risk of a transfer of the individual 110 to the emergency department.

The caregiver 115, or the companion, may also indicate on the status sheet his general feeling, namely whether the individual 110 is getting better or less well than the last time, or whether his health seems to be identical as the last time.

The computer server 120 is connected to a database 122 storing the status sheets established beforehand for a group of persons and a database 124 storing the dates of transfers of this group of persons to the emergency department.

From the status sheets and the dates of transfers of the group of persons to the emergency department, parameters of the machine learning algorithm are generated by means 126 for generating said parameters. To this end, the computer server 120 may be configured to generate said parameters.

To predict the risk of transfer to the emergency department, the data processing system 100 comprises means 128 for determining the risk factor through the analysis of a plurality of status sheets of the individual 110 thanks to the machine learning algorithm whose parameters have been generated beforehand.

As soon as the value of the risk factor exceeds a predetermined threshold, a warning is generated by means for generating 130 a warning of the data processing system 100. In particular, this warning may be a text message sent to an intervention platform 140 in order to be able to rapidly take charge of the individual 110.

For the regular filling of the status sheet, the system 100 comprises a device 150 for filling a status sheet which is generally a smartphone or a tablet used by the caregiver 115.

Advantageously, the system 100 also comprises, in the present non-limiting example of the invention, at least one sensor 155 for detecting a movement installed at the home of the individual 110 allowing detecting, according to the position of the sensor(s) 155, whether the individual stands up, whether the individual falls, whether the individual moves at his home or whether the individual leaves his home. From the data of the sensor(s), it may also be possible to determine in which room of the home is the individual 110, for example whether he is in a room, a living room, a bathroom or a kitchen.

Thus, all or part of the monitoring indicators D1 to D7 can be automatically determined.

In variants of this particular embodiment of the invention, the system comprises a camera whose data processing allows determining a movement of the individual 110. A face recognition algorithm may also be used to differentiate two individuals.

The system 100 may also comprise a device 160 allowing detecting whether an object is stored at an unusual location. The device 160 may comprise a RFID sensor allowing detecting the presence and/or the position of an object on which a RFID tag is secured.

The monitoring indicator C7 can then be automatically determined.

In variants of this particular embodiment of the invention, the device 160 is based on the combination of sensors and of NFC, instead of RFID, tags.

In order to collect the data originating from the filling device 150, the sensors 155 and/or the detection device 160 and to transmit them to the computer server 120, the system 100 also comprises a collection terminal 170 comprising wireless communication means for receiving these data.

Afterwards, the collection terminal 170 transmits the status sheet filled by the caregiver 115, and possibly partially automatically from the data originating from the sensors 155 and/or from the detection device 160, to the computer server 120 which records the status sheet associated to the individual 110 while time-stamping it.

Advantageously, the collection terminal 170 may comprise a clock allowing configuring filling and sending of the status sheet at regular intervals.

It should be highlighted that the status sheet could be filled only partially, with at least the aforementioned nine or ten monitoring indicators, namely the monitoring indicators A2, A3, A4, B2, B4, C6, D2, D4 and D7, and possibly C7. Indeed, the risk factor of a transfer of the individual 110 to the emergency department can be determined based on these nine or ten monitoring indicators.

Once four status sheets have been recorded for the individual 110, the analysis of the evolution of the monitoring indicators can be performed by the machine learning algorithm whose parameters have been generated beforehand.

In order to improve the prediction of a transfer to the emergency department in the near future, the data processing system 100 also comprises a database 180 storing the geolocated and dated epidemiological information relating to the temperature of the commune, relating to influenza-like and/or acute diarrhea illnesses.

By correlating the data of this base 180 with the status sheets of the group of persons and the transfers to the emergency department, it is thus possible to improve the generation of the parameters of the learning computer algorithm, and increase the quality of the prediction of the risk of transfer of the individual 110 to the emergency department.

In order to improve even further the determination of the risk of a transfer to the emergency department, the data processing system 100 may also comprise a database 185 storing an information sheet for each person of the group of persons for which at least one status sheet is stored in a database 122 and/or at least one date of transfer to the emergency department is stored in a database 124. Each information sheet comprising the age of the person, the classification of the person in an iso-resource group (GIR) according to the stage of his loss of autonomy, the assistance plan associated to the person and possibly his medical prescriptions. In general, the assistance plan indicates whether the person needs a homecare attendant, a meals-on-wheels delivery, a housekeeper, and possibly a technical assistance, such as a wheelchair, a cane, a walker or a healthcare bed.

It should be highlighted that the status sheets are generally recorded in the database 122.

Furthermore, as soon as the individual 110 has been transferred to the emergency department, the date of transfer of the individual 110 to the emergency department is recorded in the database 124.

An update of the parameters of the computer algorithm can then be performed while taking into account the date of transfer of the individual 110 to the emergency department.

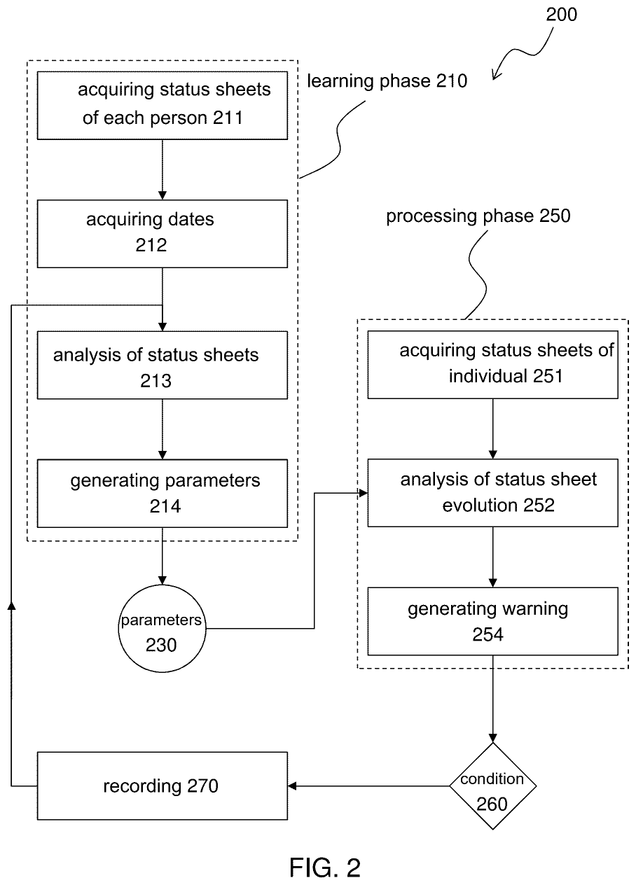

FIG. 2 illustrates, in the form of a flowchart, the data processing method 200 implemented by the data processing system 100.

The data processing method 200 comprises two main phases: a learning phase 210 and a processing phase 250.

The learning phase 210 comprises a first step 211 of acquiring a plurality of status sheets for each person of a group of persons and a second 212 one of acquiring the dates of transfers of the same group of persons to the emergency department.

Afterwards, the status sheets correlated with the dates of transfers of all or part of the persons of the group to the emergency department are analyzed during a third step 213 of the phase 210.

This analysis allows generating the parameters 230 of the machine learning algorithm during a fourth step 214.

In order to improve the determination of the parameters, the analysis performed at step 213 takes also into account, in the present non-limiting example of the invention, the geolocated epidemiological information stored in the database 180.

In other words, the learning phase of the machine learning algorithm is based on the analysis of status sheets and the corresponding dates of transfers of a group of individuals to the emergency department. During this phase, a large dataset is constructed by aggregating multiple status sheets, each containing a set of binary observational indicators, and associating them with outcome data indicating whether and when a transfer to the emergency department occurred. The algorithm processes this historical data to identify patterns and correlations between the evolution of the indicators and the likelihood of an imminent transfer. By training on this dataset, the algorithm generates and optimizes its internal parameters, enabling it to accurately assess the risk of future transfers for new individuals based on their current and past status sheets. This learning process ensures that the predictive model is tailored to the specific population and real-world scenarios encountered in the monitored group.

Afterwards, the parameters generated during step 214 are used during the processing phase 250 which comprises a first step 251 of acquiring a plurality of status sheets of the individual 110.

Afterwards, the evolution of these sheets is analyzed during a second step 252 of the machine learning algorithm whose parameters have been generated during the learning phase 210 in order to determine the value of a risk factor representative of a risk of a transfer to the emergency department in the near future.

In other words, the system updates the risk score at predefined analysis intervals by incorporating data from at least one newly filled status sheet. During the update process, the system analyzes the temporal evolution of these indicators by comparing the newly acquired status sheet with previously recorded ones. This analysis is performed using the trained machine learning algorithm. This algorithm evaluates changes in the indicators, including trends or deviations, to refine the risk score. By continuously integrating new data at regular intervals, the system ensures that the risk score remains dynamic and reflective of the individual's most recent condition.

It could be emphasized that the machine learning methods suitable for implementing the invention include supervised learning algorithms capable of analyzing the evolution of binary indicators across multiple status sheets. These methods may include decision tree-based algorithms, such as Random Forest or Gradient Boosted Trees, which are well-suited for handling categorical data and identifying intricate patterns in the binary indicators. Additionally, support vector machines (SVM) can be employed to classify the risk of a symptom or event by finding optimal hyperplanes in the feature space. Neural networks, particularly shallow architectures, may also be utilized to model non-linear relationships between the indicators and the predicted risk. Ensemble methods, which combine the outputs of multiple models to enhance predictive accuracy, are advantageous in this context. The choice of algorithm is guided by the need for interpretability, computational efficiency, and the ability to process large datasets of binary observational indicators while maintaining robust predictive performance.

When the risk factor exceeds a predetermined threshold, an alert is generated during a fourth step 254.

The system is configured to transmit an alert to a monitoring platform or healthcare professional when the calculated risk factor exceeds a predetermined threshold. This alert can be generated in various forms, such as a text message, email, or push notification, and is typically sent through secure communication channels to ensure data privacy and compliance with healthcare regulations. The alert includes relevant information, such as the individual's identification, the calculated risk factor, and the specific indicators contributing to the elevated risk. This enables the healthcare professional or monitoring platform to promptly assess the situation and take appropriate action, such as scheduling a home visit, initiating a teleconsultation, or mobilizing emergency services. By providing timely and actionable insights, the system facilitates early intervention, potentially preventing the individual's condition from worsening and reducing the likelihood of emergency department admission.

The alert can also be generated by analyzing the temporal evolution of the risk factor over a predefined period. This involves monitoring changes in the calculated risk factor across multiple status sheets recorded at distinct time points. By evaluating trends, such as a steady increase or sudden spike in the risk factor, the system can identify patterns indicative of an imminent transfer to the emergency department. This temporal analysis enhances the predictive accuracy by considering not only the current risk factor but also its progression over time, allowing for earlier and more reliable detection of high-risk situations. When the temporal trend exceeds a predetermined threshold, an alert is automatically triggered and transmitted to a monitoring platform or healthcare professional, enabling timely intervention.

If the individual 110 is admitted to the emergency department, represented by the condition 260, the method 200 may advantageously update the parameters of the machine learning algorithm.

To this end, the method 200 comprises a step 270 of recording the status sheets and the date of transfer of the individual 110 to the emergency department respectively in the database 122 and 124.

Steps 213 and 214 of the learning phase are then performed again to update the parameters of the computer algorithm.

FIGS. 3A-3F represent examples of comparison of the results obtained by basing the analysis on different combinations of indicators.

In other words, when an analysis is based on a determined combination of indicators, the steps of analyzing 213 and generating 214 the parameters of the algorithm executed during the learning phase 210 are performed while considering only the indicators of this combination on the status sheets. If the status sheet comprises other indicators, these are not considered, which amounts to the status sheet comprising only the determined combination of indicators.

The step of determining the value of the risk factor through the analysis 252 of the status sheets of the individual 110 executed during the processing phase 250 is also performed while considering only the indicators of the determined combination.

FIGS. 3A-3F comprise six graphs, each corresponding to a combination of indicators.

Each graph comprises two curves ROC (acronym of “Receiver Operating Characteristic”) allowing characterizing the performance of a binary classifier by representing the true positive rate, that is to say the fraction of positives that are effectively detected, as a function of the false positive rate, fraction of the negatives that are wrongly detected.

In each graph, the true positive rate, indicated in FIG. 3 by the term “True Positive Rate”, is in the ordinates, whereas the false positive rate, indicated in FIG. 3 by the term “False Positive Rate”, is in the abscissas.

Moreover, the curve “TRAIN ROC” illustrates the learning phase 210 during which the parameters of the algorithm according to the analysis of the status sheets stored in the database 122.

In turn, the curve “TEST ROC” illustrates the processing phase 250 during which a value representative of a risk of a transfer of the individual 110 to the emergency department is calculated. To establish the curve, this analysis is performed on a plurality of individuals 110, selected randomly, in order to calculate the predictive performance represented by the surface located under the curve “TEST ROC” by comparing the obtained value of the risk factor with the actual transfer to the emergency department stored in the database 124.

To this end, the curves “TRAIN ROC” and “TEST ROC” have been calculated in the present example, by defining two cohorts from the actual data stored in the database 124. The first cohort, corresponding to 70% of the persons registered in the databases 122 and 124, is used to establish the curve “TRAIN ROC”. Whereas the second cohort, corresponding to 30% of the persons registered in the databases 122 and 124, is used to establish the curve “TEST ROC”.

The graph represented in FIG. 3A corresponds to the combination of the nine indicators:
A2. the individual has difficulties in breathing;
A3. the individual is feverish;
A4. the individual has pains;
B2. the individual is not very communicative;
B4. the individual has contacts or visits with his entourage;
C6. the individual is sad;
D2. the individual moves at his home;
D4. the individual prepares his meals;
D7. the individual falls.

It should be highlighted that the slope at the origin of the curve 310, corresponding to the curve “TEST ROC” of FIG. 3A, is almost vertical, which sets out an advantage of the use of this combination of nine indicators in the data processing method 200 for the determination of the risk factor of an imminent transfer to the emergency department. Indeed, this vertical slope indicates that the transfer of most of the first individuals 100 object of the analysis to the emergency department will be effectively detected. Thus, they can be managed very quickly by a healthcare service.

The graph represented in FIG. 3B corresponds to the combination of the five indicators:
A3. the individual is feverish;
A4. the individual has pains;
B4. the individual has contacts or visits with his entourage;
D2. the individual moves at his home;
D4. the individual prepares his meals.

The obtained predictive performance is in the range of 54%, with a slope that is also vertical at the origin.

The graph represented in FIG. 3C corresponds to the combination of the nine indicators:
A4. the individual has pains;
B1. the individual is indifferent;
B2. the individual is not very communicative;
C1. the individual refuses help with toileting;
C2. the individual does not recognize the companion;
C6. the individual is sad;
D7. the individual falls;
E1. the caregiver is sad;
E2. the caregiver is exhausted.

The predictive performance obtained with this combination is 53%.

The graph represented in FIG. 3D corresponds to the combination of the eight indicators:
A1. the individual has swollen legs;
A2. the individual has difficulties in breathing;
A3. the individual is feverish;
B2. the individual is not very communicative;
B4. the individual has contacts or visits with his entourage;
D2. the individual moves at his home;
D4. the individual prepares his meals;
D7. the individual falls.

The predictive performance obtained with this combination is 52%.

The graph represented in FIG. 3E corresponds to the combination of the eight indicators:
A2. the individual has difficulties in breathing;
A4. the individual has pains;
C1. the individual refuses help with toileting;
C6. the individual is sad;
C8. the individual seems tired;
D2. the individual moves at his home;
D4. the individual prepares his meals;
D7. the individual falls.

The predictive performance obtained when basing the analysis on this combination is 53%.

The graph represented in FIG. 3F corresponds to the combination of the six indicators:
A1. the individual has swollen legs;
A2. the individual has difficulties in breathing;
A3. the individual is feverish;
A4. the individual has pains;
B3. the individual lives alone since at least seven days;
D7. the individual falls.

The predictive performance obtained when basing the analysis on this combination is 56%.

### Variant of Embodiment of the Invention

In a variant embodiment of the invention, the status sheet further comprises at least one physiological parameter, such as body temperature, heart rate, blood pressure, oxygen saturation, or body weight. These physiological data are typically collected using a sensor operated by a caregiver or assistant visiting the monitored individual 110, without requiring advanced medical expertise. The integration of physiological parameters into the status sheet allows the system to enhance its predictive capabilities by combining observational indicators with objective health measurements. This hybrid approach enables the machine learning algorithm to consider both behavioral and physiological changes, potentially improving the accuracy and reliability of risk assessment for emergency department transfer. Furthermore, the use of user-friendly sensors ensures that data collection remains accessible and minimally invasive, supporting regular monitoring in a home environment.

The described approach emphasizes the use of binary observational indicators as the primary data source for the machine learning algorithm, with physiological data playing a secondary or optional role. Binary indicators, such as “Yes” or “No” responses to predefined questions about an individual's health, behavior, or environment, are central to the described method due to their simplicity, accessibility, and ease of collection without requiring specialized medical equipment or expertise. These indicators enable a robust and scalable analysis of trends and patterns across multiple status sheets, forming the foundation of the algorithm's predictive capabilities. While physiological data, such as heart rate or body temperature, may be optionally incorporated to enhance prediction accuracy, their role is deliberately weighted lower in the algorithm to maintain the system's focus on non-invasive, observational data. This prioritization ensures that the described method remains minimally intrusive, cost-effective, and widely applicable, particularly for elderly individuals living autonomously in non-medicalized environments.

In others words, the machine learning algorithm is specifically designed to assign greater weight to binary observational indicators than to physiological data when calculating the risk score. This prioritization is implemented either through explicit weighting coefficients within the model or by selecting algorithmic architectures—such as decision trees or ensemble methods—that naturally emphasize categorical and binary features. The binary indicators, which reflect observable aspects of the individual's health, behavior, and environment, are considered the core predictive elements due to their accessibility and reliability in non-medicalized settings. Physiological data, when available, could be incorporated as supplementary features and their influence on the final risk assessment is deliberately limited to ensure that the system remains robust even in the absence of such data. This approach guarantees that the predictive model remains focused on the primary, non-invasive data sources, thereby maintaining the invention's intended simplicity, scalability, and broad applicability.

### Example of Another Particular Embodiment of the Invention

A similar method based on the trend analysis of status sheet comprising binary observational indicators relating to health, social interaction, behavior, and/or physical or sensory capabilities of the individual, without any physiological data, can be used to predict outcome of symptoms in a monitored individual, such as a risk of falling, swollen legs, malnutrition, or a risk of depression.

The prediction of a symptom, such as a risk of falling, swollen legs, malnutrition, or a risk of depression, is notably carried out in a surprising manner by analyzing the evolution of status sheets of the individual, each status sheet being established at distinct times and including a plurality of monitoring indicators for the individual, each indicator having a value chosen between two binary values, generally a positive value (“Yes”) and a negative value (“No”).

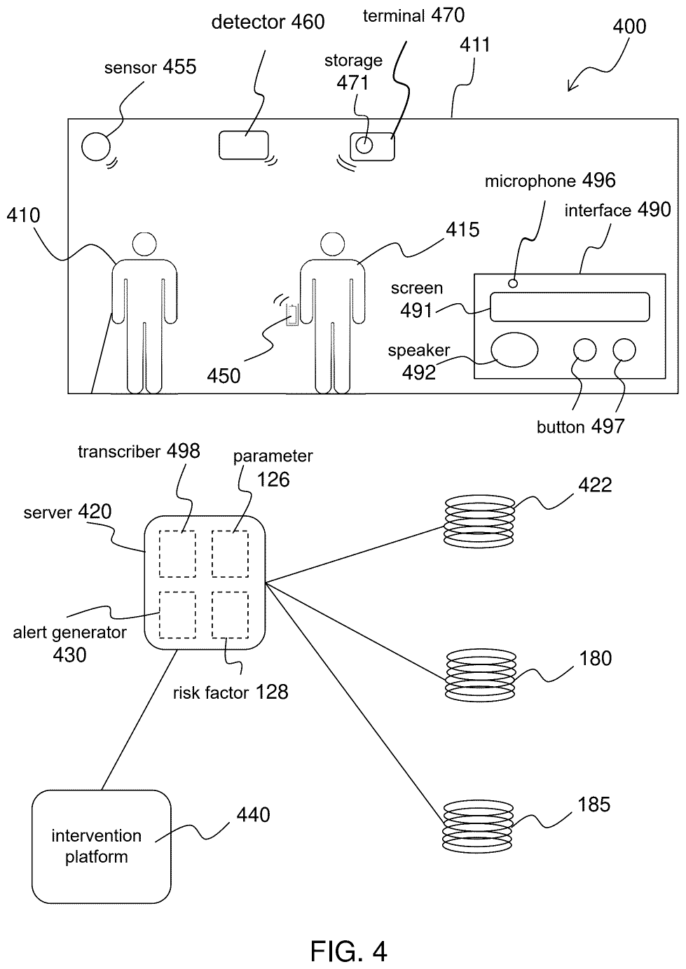

FIG. 4 is a simplified diagram of a system 400 for remote monitoring of an individual 410, potentially elderly, in an everyday environment, for example, the home 411 of the individual 410. The remote monitoring system for this purpose comprises a computer server 420 equipped with a microprocessor and computer memory in which a machine learning algorithm configured by parameters to predict the onset of at least one symptom in the individual 410 is stored.

It should be noted that the machine learning algorithm is generally chosen from machine learning techniques more commonly known by the English term “machine learning,” such as, for example, a “random forest” type algorithm.

It should be noted that the status sheets, notably devoid of physiological data of the individual 410, can be filled out by anyone. Each status sheet can thus be filled out, for example, by a caregiver 415 visiting the individual 410. It should be noted that a caregiver of the individual 410 can fill out the status sheet instead of the caregiver 415.

Each status sheet comprises a list of monitoring indicators generally chosen from the following global list of monitoring indicators:
health-related indicators such as:
   A1. the individual has swollen legs;
   A2. the individual has difficulty breathing;
   A3. the individual is feverish;
   A4. the individual has pain;
relational indicators such as:
   B1. the individual is indifferent;
   B2. the individual is not very communicative;
   B3. the individual has been living alone for at least seven days;
   B4. the individual has contact or visits with their surroundings;
behavioral indicators such as:
   C1. the individual refuses help with toileting;
   C2. the individual does not recognize the caregiver;
   C3. the individual forgets when the caregiver visited;
   C4. the individual communicates incoherently;
   C5. the individual is aggressive;
   C6. the individual is sad;
   C7. the individual puts objects in inappropriate places;
   C8. the individual seems tired;
   C9. the individual refuses the caregiver's intervention;
   C10. the individual gets dressed;
indicators representative of the individual's physical and sensory abilities such as:
   D1. the individual gets up;
   D2. the individual moves around their home;
   D3. the individual takes care of their personal hygiene;
   D4. the individual prepares their meals;
   D5. the individual leaves their home;
   D6. the individual eats;
   D7. the individual falls.

These indicators can be accompanied by indicators related to the caregiver such as:
E1. the caregiver is sad;
E2. the caregiver is exhausted.

It should be noted that each indicator is representative of a state and that a similar or equivalent formulation of one or more indicators could be used without significantly altering the results obtained. For example, indicator B3 related to the individual's loneliness could be formulated as “the individual feels lonely.”

It should be noted that the list of binary indicators is substantially identical to that of the first embodiment, except for the addition of a new indicator C10, which may also be incorporated into the first embodiment.

The joint analysis of all or part of the indicators of each status sheet by the machine learning algorithm makes it possible to predict an upcoming symptom such as a fall, depression, malnutrition, or swollen legs. The parameters of the machine learning algorithm are thus configured during a learning phase based on statistical analysis of the evolution of the indicators over time for the plurality of people.

For example, an upcoming fall can be predicted by the joint analysis of all or part of the following indicators:
C8. the individual seems tired;
C2. the individual does not recognize the caregiver;
D4. the individual prepares their meals;
D5. the individual leaves their home;
C3. the individual forgets when the caregiver visited.

Similarly, malnutrition can be predicted by the joint analysis of all or part of the following indicators:
C10. the individual gets dressed;
C8. the individual seems tired;
D5. the individual leaves their home;
A4. the individual has pain;
D4. the individual prepares their meals.

Depression can be predicted by the joint analysis of all or part of the following indicators:
A4. the individual has pain;
C8. the individual seems tired;
C3. the individual forgets when the caregiver visited;
D5. the individual leaves their home.

Finally, the onset of swollen legs can be predicted by the joint analysis of all or part of the following indicators:
A4. the individual has pain;
D5. the individual leaves their home;
C8. the individual seems tired;
D4. the individual prepares their meals;
B4. the individual has contact or visits with their surroundings.

To improve the prediction of each of these symptoms, other indicators can be analyzed.

For example, to improve the prediction of an upcoming fall, all or part of the following indicators can be analyzed jointly:
C8. the individual seems tired;
C2. the individual does not recognize the caregiver;
D4. the individual prepares their meals;
D5. the individual leaves their home;
C3. the individual forgets when the caregiver visited;
C7. the individual puts objects in inappropriate places;
B4. the individual has contact or visits with their surroundings.

Similarly, to improve the prediction of malnutrition, all or part of the following indicators can be analyzed jointly:
C10. the individual gets dressed;
C8. the individual seems tired;
D5. the individual leaves their home;
A4. the individual has pain;
D4. the individual prepares their meals;
B4. the individual has contact or visits with their surroundings;
C2. the individual does not recognize the caregiver.

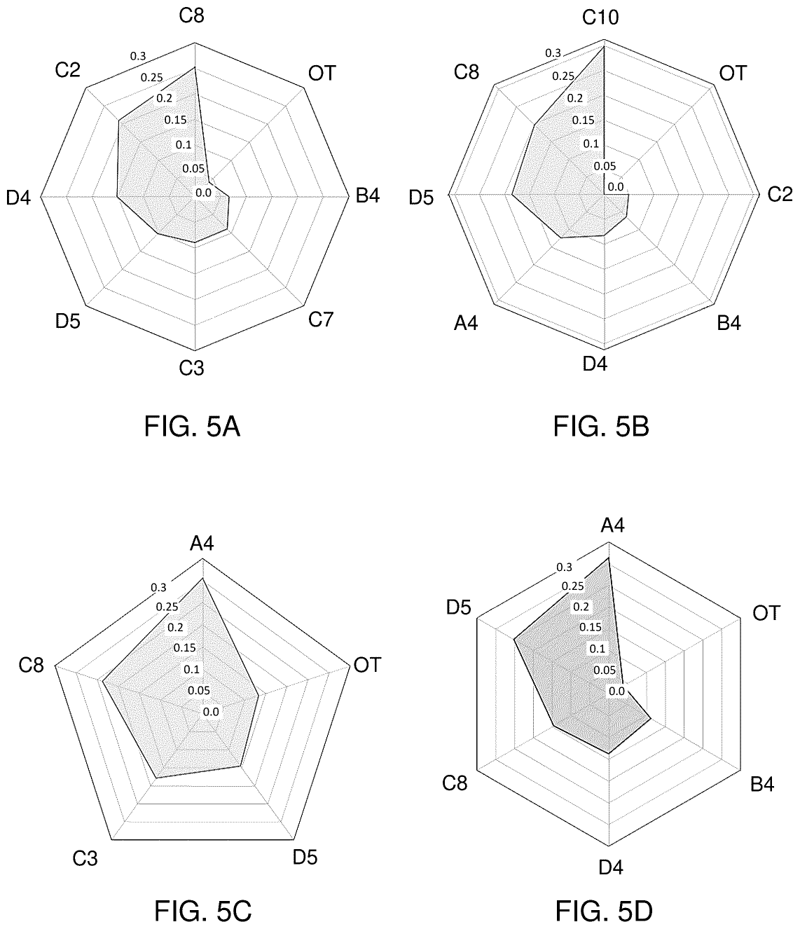

It should be noted that the indicators have been ranked, in this non-limiting example of the invention, by order of importance for each symptom, as illustrated by the radar charts in FIGS. 5A to 5D. For each radar chart, the relative weight of the corresponding indicator in the prediction is indicated. The other indicators of the status sheet are grouped under the term OT (for the English term “others”).

All or part of the monitoring indicators of the status sheet can be associated with a sub-indicator indicating a precision related to said monitoring indicator, namely an evolution of the state that is the object of said indicator compared to the last filling of the status sheet. The sub-indicator is chosen between three values generally corresponding to an improvement in the state (e.g., “better”), stabilization of the state (e.g., “same”), and deterioration of the state (e.g., “worse”). This sub-indicator makes it possible to provide nuance regarding the indicator whose value has not been modified between two successively filled status sheets.

A sub-indicator can, for example, be associated with monitoring indicators A1 to A4, B1, C8, D2, D6, and/or D7, which can further improve the prediction.

The computer server 420 is connected to a database 422 storing the status sheets established previously for a group of people.

From the status sheets of the group of people, parameters of the machine learning algorithm are generated by means for generating said parameters by analyzing the combined evolution of the indicators over time. For this purpose, the computer server 420 can be configured to generate said parameters.

The remote monitoring system 400 comprises means for predicting a symptom by analyzing a plurality of status sheets of the individual 410 using the machine learning algorithm whose parameters have been previously generated. In the present embodiment, the machine learning algorithm provides as output at least one risk value associated with a given symptom.

As soon as a symptom is predicted, for example when the risk value associated with said symptom exceeds a predetermined threshold, an alert is generated by means 430 for generating an alert of the remote monitoring system 400. This alert can notably be a text message sent to an intervention platform 440 in order to be able to take care of the individual 410 quickly and avoid them ending up in the emergency room.

For the regular filling of the status sheet, the caregiver 415 uses the device 450 for filling out a status sheet which is generally a smartphone or a tablet.

Advantageously, the system 400 also comprises, in this non-limiting example of the invention, at least one motion detection sensor 455 installed in the individual's home 410, making it possible to detect, depending on the position of the sensor(s) 455, if the individual gets up, if the individual falls, if the individual moves around their home, or if the individual leaves their home. From the data of the sensor(s), it may also be possible to determine in which room of the home the individual 410 is located, for example, if they are in a bedroom, living room, bathroom, or kitchen.

Thus, all or part of the monitoring indicators D1 to D7 can be determined automatically.

In variants of this particular embodiment of the invention, the system comprises a camera whose data processing makes it possible to determine a movement of the individual 410. A facial recognition algorithm can also be used to differentiate between two individuals.

The system 400 can also comprise a device 460 for detecting if an object is placed in an unusual location. The device 460 can comprise an RFID sensor for detecting the presence and/or position of an object on which an RFID chip is attached.

The monitoring indicator C7 can then be determined automatically.

In variants of this particular embodiment of the invention, the device 460 is based on the combination of NFC sensors and chips instead of RFID.

To collect the data coming from the filling device 450, the sensors 455, and/or the detection device 460 and to transmit them to the computer server 420, the system 400 also comprises a collection terminal 470 including wireless communication means for receiving this data.

The collection terminal 470 then transmits the status sheet filled out by the caregiver 415 or partly automatically from the data coming from the sensors 455 and/or the detection device 460, to the computer server 420, which records the status sheet associated with the individual 410 by timestamping it.

Advantageously, the collection terminal 470 can comprise a clock for configuring the filling and sending of the status sheet at regular intervals. The collection terminal 470 can also comprise an electrical energy storage device 471 to make it autonomous.

It should be noted that the status sheet can be filled out only partially, with at least the indicators corresponding to the detection of a particular symptom.

As soon as a minimum of status sheets, for example four, are recorded for the individual 410, the analysis of the evolution of the monitoring indicators can be carried out by the machine learning algorithm whose parameters have been previously generated.

The status sheet can also be filled out through an interaction of the individual 410 with an interface 490 of the remote monitoring system 400, distinct or not from the filling device 450.

For this purpose, the interface 490 comprises a device for providing at least one question to the individual 410 visually, for example via a screen 491, or audibly, for example via a speaker 492.

The individual 410 can respond to each question via the interface 490, which has a device 495 for recording the response(s). The recording device can be, for example, a microphone 496 or a plurality of buttons 497 (for example, two buttons: “yes” and “no”).

A device 498 for transcribing each response can then be used to determine the value of at least one indicator of the status sheet being filled out.

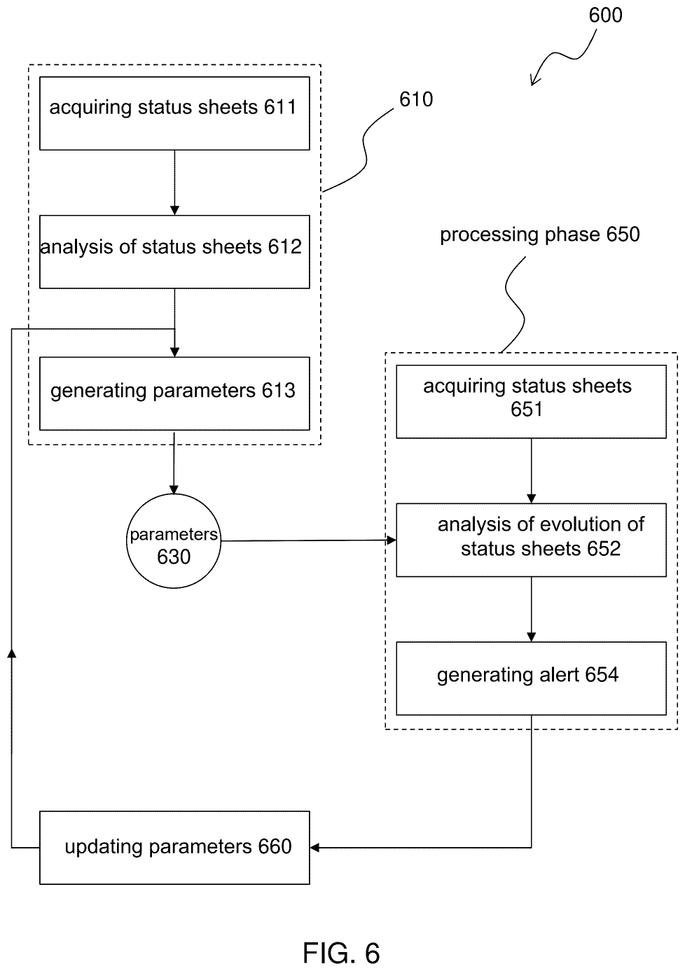

FIG. 6 illustrates in the form of a synoptic diagram the data processing method 600 implemented by the remote monitoring system 400.

The data processing method 600 comprises two main phases: a learning phase 610 and a processing phase 650.

The learning phase 610 comprises a first step 611 of acquiring a plurality of status sheets for each person in a group of people in the database 422.

The combined evolution of the indicators of the status sheets of all or part of the people in the group is then analyzed during a second step 612 of the phase 610, notably with respect to the onset of a symptom that can be identified through the indicators of the status sheets. For example, the evolution of indicators C8, C2, D4, D5, and C3 is analyzed in the status sheets of each individual preceding those where a fall (indicator D7) is observed for these individuals.

The same applies to malnutrition, which can be observed through indicator D6, for sadness/depression through indicator C6, and for swollen legs through indicator A1.

This analysis makes it possible to generate the parameters 630 of the machine learning algorithm during a third step 613.

The parameters 630 generated during step 613 are then used during the processing phase 650, which comprises a first step 651 of acquiring a plurality of status sheets of the individual 410.

The evolution of these sheets is then analyzed during a second step 652 by the machine learning algorithm whose parameters have been generated during the learning phase 610 in order to determine at least one risk value associated with a particular symptom within a predetermined near future time frame.

When at least one risk value exceeds a predetermined threshold, an alert is generated during a fourth step 654, indicating the symptom associated with the risk value.

It should be noted that the method 600 can advantageously update the parameters of the machine learning algorithm regularly by supplementing the database 422 with the sheets of the individual during a step 660.

Steps 613 and 614 of the learning phase are then carried out again to update the parameters of the algorithm.

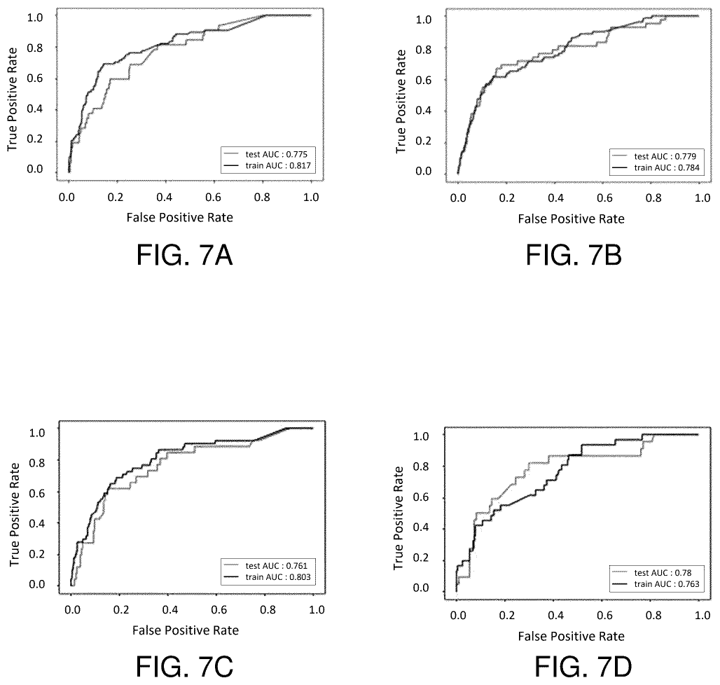

FIGS. 7A-7D represent the ROC curves obtained respectively for a risk of falling, a risk of malnutrition, a risk of swollen legs, and a risk of depression for an individual who has not had these symptoms previously.

On each graph, the true positive rate, indicated by the English term “True Positive Rate,” is on the y-axis, while the false positive rate, indicated by the English term “False Positive Rate,” is on the x-axis.

Furthermore, the “TRAIN” curve illustrates the learning phase 610 during which the parameters of the algorithm are generated based on the analysis of the status sheets stored in the database 422.

The “TEST” curve, on the other hand, illustrates the processing phase 650 during which a value representative of a risk of onset of a symptom in the individual 410 is calculated. To establish the curve, this analysis is carried out on a plurality of individuals 410, chosen randomly, in order to calculate the predictive performance represented by the area under the “TEST” curve by comparing the obtained risk value with the onset of the symptom observed in the status sheet (for example: D7 for falling, D6 for malnutrition, C6 for depression, A1 for swollen legs).

For this purpose, the “TRAIN” and “TEST” curves have been calculated in the present example by defining two cohorts. The first cohort, corresponding to 70% of the people registered in the database 422, is used to establish the “TRAIN” curve. While the second cohort, corresponding to 30% of the people registered in the database 422, is used to establish the “TEST” curve.

### Generic Embodiment of the Data Processing Method of the Invention

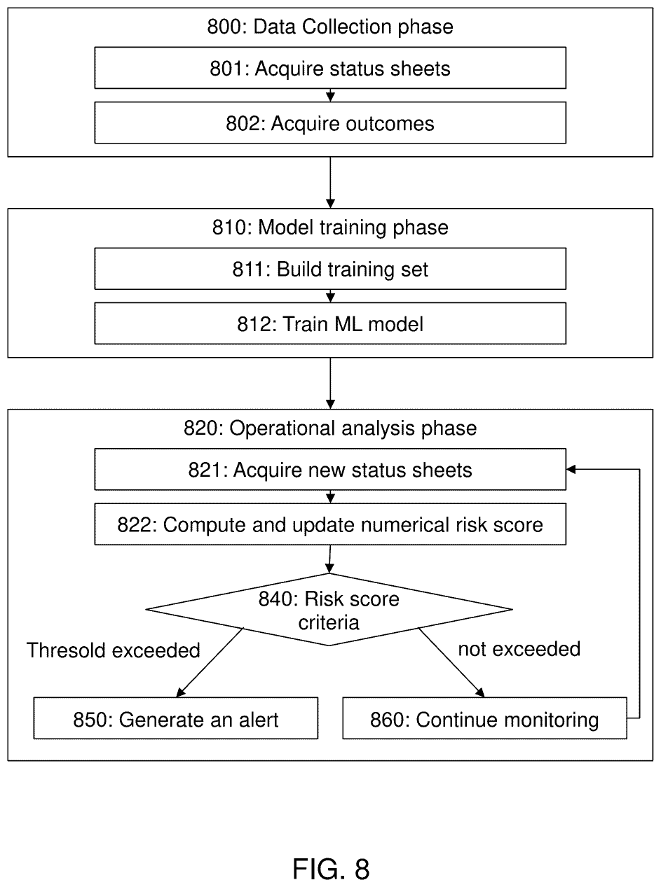

FIG. 8 illustrates, in the form of a flowchart, a generic embodiment of the data processing method as implemented in the previously described examples. The flowchart schematically represents the successive functional phases that may be carried out in different ways depending on the embodiment. These phases include the acquisition of observational data and optional physiological parameters, the training of a predictive model, the operational analysis of the monitored individual's status sheets, the computation and updating of a risk score, and the generation of an alert when a predetermined threshold is exceeded.

In a first step 801, corresponding to the data collection phase, the system acquires a plurality of dated status sheets for a group of individuals. Each status sheet includes a plurality of binary observational indicators relating for example to the health condition of the individual (such as the presence of pain, fever, or difficulties in breathing), to social interaction (such as whether the individual is communicative or receives visits from relatives), to behavior (such as sadness, aggression, or storage of objects in inappropriate places), and/or to physical or sensory capabilities (such as the ability to prepare meals, move inside the home, or occurrences of falls). These indicators are deliberately simple, with values generally limited to “Yes” or “No,” so that the status sheets can be completed by a caregiver, an assistant, or even a relative without requiring medical training. In certain embodiments, the status sheet also comprises at least one physiological parameter, such as blood pressure, heart rate, body temperature, oxygen saturation, or body weight, collected with the aid of non-invasive sensors. These status sheets are correlated with outcome data representing hospital admission events or the onset of a specific symptom such as malnutrition, depression, swollen legs, or a fall. These outcomes are acquired during a second step 802 of the data collection phase 800.

The method then enters the model training phase 810. In step 811, the acquired data are assembled into a training dataset, combining the dated status sheets, the outcome data, and a set of “negative” status sheets corresponding to individuals for whom no imminent transfer or symptom occurrence has been observed. This ensures that the dataset contains both positive and negative examples, enabling the algorithm to learn discriminative patterns. In step 812, the dataset is processed by a machine learning algorithm, which may be selected among decision-tree based models such as Random Forests or Gradient Boosted Trees, support vector machines, or neural networks. Depending on the embodiment, the trained model may be configured as a classifier that exclusively considers binary observational indicators, as a hybrid model giving greater predictive weight to observational indicators while integrating physiological parameters as secondary features, or as a classifier specifically oriented toward predicting the onset of a particular symptom. The output of this phase is a set of optimized parameters that encode the correlations between the temporal evolution of indicators and the risk of an adverse event.

Once the model has been trained, the method proceeds to the operational analysis phase 820. In step 821, the system acquires new status sheets of the monitored individual at distinct time points, for example every week or several times per week. Step 822 consists in extracting temporal attributes from these new status sheets. These attributes may include simple differences in the value of each indicator compared with previous sheets, as well as more elaborate descriptors such as smoothed trends over several recordings. In embodiments that include physiological parameters, this step also generates variables representing the short-term evolution of these parameters in order to enrich the prediction. In step 822, the trained model is applied to the new data in order to compute a numerical risk score. This score represents the probability of either a hospital transfer or the onset of a symptom within a predetermined time window, generally the following seven days. The model ensures that changes in the indicators are correctly interpreted in light of past data, so that both abrupt deteriorations and gradual worsening trends are taken into account.

In step 822, the computed risk score is updated at predefined analysis intervals. The updating process consists in recalculating the risk score each time a new status sheet is available, thus ensuring that the system continuously reflects the most recent condition of the monitored individual. Decision step 840 evaluates the value of the updated score as well as its temporal evolution over several recordings. If the score, or its trend, exceeds a predetermined threshold, step 850 generates an alert. The alert may be transmitted in the form of a secure message to a monitoring platform or directly to a healthcare professional, and may include information identifying the individual, the current risk score, and the specific indicators contributing most strongly to the prediction. If the threshold is not exceeded, step 860 continues monitoring and the method loops back to step 821, awaiting new status sheets to maintain continuous monitoring.

The flowchart of FIG. 8 thus provides a generic representation of the invention, encompassing the three embodiments described herein. In a first embodiment, the method relies solely on binary observational indicators, thereby ensuring simplicity and accessibility of data collection. In a second embodiment, the method integrates both observational indicators and physiological parameters, the model giving greater weight to the observational data in order to maintain robustness even if physiological measurements are missing or inconsistent. In a third embodiment, the method is oriented toward the prediction of specific symptoms such as falls, malnutrition, or depression, the risk score being calculated for each symptom separately.

It could be emphasized that when the calculated risk score or its temporal trend exceeds a predetermined threshold, the system might also be configured to automatically instantiate a preventive care workflow. This workflow may comprise at least one of: (i) automatically creating and assigning a home-visit task in a digital scheduling module, the assignment including the selection of an available slot within a predefined time window of 24 to 72 hours; (ii) automatically generating a one-time teleconsultation session link transmitted simultaneously to the designated healthcare professional and to a caregiver or family member of the monitored individual; (iii) automatically transmitting and recording a time-stamped confirmation of the scheduled intervention in a secure care-coordination log; and (iv) in cases of elevated risk, automatically issuing a request for non-emergency medical transportation to ensure preventive transfer to an appropriate healthcare facility prior to the onset of acute symptoms.

Furthermore, the methods and systems of the present invention are intended for remote monitoring in non-medicalized environments, such as private homes, assisted living facilities, or nursing homes, where medical staff are not continuously present like in a hospital. They are not intended to substitute for in-person triage performed in a hospital emergency department.

The method implemented by the system relies on supervised machine learning algorithms capable of processing heterogeneous data, including categorical observational indicators and continuous physiological parameters. The training dataset comprises both positive cases, corresponding to individuals for whom a hospital transfer or the occurrence of a symptom has been observed within a predetermined time window, and negative cases, corresponding to individuals for whom no such event has occurred. By learning discriminative patterns from both types of cases, the trained model is able to provide reliable predictions for unseen individuals while reducing the number of false positives.

In some embodiments, the predictive algorithm is based on decision tree ensembles, such as Random Forests or Gradient Boosted Trees. These algorithms are well suited to structured medical data, as they can directly handle binary variables, missing values, and non-linear relationships between features. Random Forests, by averaging the results of a large number of independent trees, provide robustness to noise and variability in the data. Gradient Boosted Trees, by contrast, progressively refine the prediction by iteratively correcting the errors of previous models, leading to high predictive performance.

In other embodiments, the system may implement regularized linear models such as logistic regression or support vector machines. Logistic regression with L1 or L2 regularization not only provides stable predictive performance but also allows automatic selection of the most relevant indicators by reducing the weight of redundant or less informative variables. Support vector machines, on the other hand, are particularly effective when the number of features is large compared to the number of available training samples, as is often the case in healthcare applications where many indicators are monitored but relatively few ground-truth events are recorded.

In further embodiments, the system may be configured with neural network architectures. A simple multilayer perceptron can capture complex, non-linear correlations between observational indicators and outcomes. More advanced recurrent architectures, such as networks with memory units, can take into account the temporal sequence of the status sheets and detect patterns of deterioration spread across several consecutive recordings. This makes it possible to detect both sudden changes and progressive declines in the monitored individual's condition.

The system may also employ hybrid architectures that combine several models in parallel. For example, a first model may be trained primarily on binary observational indicators, while a second model focuses on continuous physiological parameters. Their outputs can then be aggregated through weighted voting or stacking, the system giving greater weight to the observational indicators to ensure robustness in cases where physiological measurements are missing or subject to measurement noise.

Regardless of the chosen algorithm, the system may also incorporate explainability mechanisms to make the results interpretable for healthcare professionals. For example, tree-based models can provide feature importance scores that highlight which indicators contributed most to the calculated risk, while linear models can produce coefficients directly indicating whether a given indicator increases or decreases the risk. This interpretability ensures that alerts generated by the system are transparent, understandable, and actionable.

### EXAMPLES

The invention will now be further illustrated by way of non-limiting examples. These examples describe implementations of the system in real-world environments and the results obtained from clinical evaluations. The examples are provided to demonstrate the practical operation and effectiveness of the invention, in particular the ability of the machine learning algorithm to predict adverse health events and to enable preventive interventions.

It will be understood that the following examples are merely illustrative, and that variations in parameters, implementation conditions, and datasets may be made without departing from the scope of the invention as defined in the claims.

In a first real-world implementation, the prediction algorithm was applied to older individuals living at home. Alerts generated by the system were transmitted to a coordinating nurse, who arranged a preventive intervention when appropriate. The results showed that emergency department (ED) visits within 14 days of an alert were significantly reduced when alerts were followed by an intervention. Only 1.5% of alerts followed by an intervention resulted in an ED visit, compared to 13.1% of alerts without intervention. The calculated odds ratio (0.10; 95% CI: 0.02-0.43) indicates a statistically significant effect. These results demonstrate that use of the system substantially reduces unplanned ED visits.

A second study conducted in assisted living facilities confirmed these findings. Among 92 alerts, 29 hospitalizations occurred. When alerts were followed by a healthcare intervention (n=46), only 4 hospitalizations (14%) were observed, compared to 25 hospitalizations (86%) when alerts were not followed by intervention (n=46). Overall, 91% of alerts followed by intervention did not lead to hospitalization within 14 days. This confirms that proactive interventions triggered by the system's alerts markedly reduce the probability of avoidable hospitalizations.

A comparative evaluation further demonstrated the effectiveness of the system relative to a control group without alerts. A total of 792 patient episodes were analyzed (66 with alerts and 726 without alerts). In the alert group, hospitalizations occurred in only 5% of the cases when alerts were followed by intervention (1 of 21), while 22% of alerts without intervention led to hospitalization. In the no-alert group, 0.6% of episodes resulted in hospitalization, but without any opportunity for anticipation or prevention. Furthermore, in terms of overall health outcomes, comparison between the intervention group and the control group showed a 33% reduction in hospitalization rates, as well as a 67% reduction in deaths during the study period. These results illustrate the ability of the system to improve patient management, anticipate deteriorations, and reduce adverse outcomes.

1. A computer-implemented method executed by a processor for assessing, within a predetermined future time window, a risk of a hospital transfer of a monitored individual, the method comprising:
a data collection phase, comprising:
acquiring, for a group of individuals, a plurality of dated status sheets, each dated status sheet comprising at least four binary observational indicators, each binary observational indicator relating to at least one of: health, social interaction, behavior, and physical or sensory capabilities of the individual, without any physiological parameter; and
acquiring outcome data relating to hospital admission events for at least some individuals of the group; and
a model training phase, comprising:
building a training dataset combining: the plurality of dated status sheets acquired, the outcome data, and a set of negative status sheets from individuals without imminent transfer; and
training, by the processor, a model of a machine learning algorithm on the training dataset to provide a trained model;
an operational analysis phase, comprising:
acquiring, for the monitored individual, a plurality of new status sheets established at distinct time points;
applying the trained model to compute a numerical risk score of the hospital transfer for the monitored individual;
updating the numerical risk score of the monitored individual at predefined analysis intervals based on at least a new status sheet;
automatically generating an alert when the numerical risk score or a temporal trend of the numerical risk score of the monitored individual exceeds a threshold and transmitting the alert to a monitoring platform or a healthcare professional.

2. The method of claim 1, wherein said at least four binary observational indicators are selected among:
indicators related to a health condition of the individual, comprising:
A1. the individual has swollen legs;
A2. the individual has difficulties in breathing;
A3. the individual is feverish; and
A4. the individual has pains;
relational-type indicators, comprising:
B1. the individual is indifferent;
B2. the individual is not very communicative;
B3. the individual lives alone since at least seven days; and
B4. the individual has contacts or visits with his entourage;
behavioral-type indicators, comprising:
C1. the individual refuses help with toileting;
C2. the individual does not recognize the companion;
C3. the individual forgets when the companion has come by;
C4. the individual communicates inconsistently;
C5. the individual is aggressive;
C6. the individual is sad;
C7. the individual stores objects in inappropriate locations;
C8. the individual seems tired; and
C9. the individual refuses the intervention of the companion; and
indicators representative of the physical or sensory capabilities of the individual, comprising:
D1. the individual stands up;
D2. the individual moves at his home;
D3. the individual performs personal hygiene;
D4. the individual prepares his meals;
D5. the individual leaves his home;
D6. the individual eats; and
D7. the individual falls.

3. The method of claim 2, wherein said each status sheet comprises at least nine binary observational indicators comprising A2, A3, A4, B2, B4, C6, D2, D4, and D7, and optionally C7.

4. The method of claim 1, wherein each binary observational indicator is associated with either an improvement sub-indicator, a stabilization sub-indicator, or a degradation sub-indicator.

5. The method of claim 1, wherein at least one binary observational indicator is automatically populated using a sensor selected from: a motion detection sensor, a weight sensor, a radio frequency identification (RFID) or an near-field communication (NFC) sensor, the RFID sensor and NFC sensor cooperating with a tag secured to a monitored object.

6. The method of claim 1, wherein the alert comprises at least one of: a text message, an email and a push notification transmitted through a secure communication channel including an identification of the monitored individual and binary observational indicators contributing to the numerical risk score.

7. The method of claim 1, wherein the machine learning algorithm is selected from a Random Forest classifier, a Gradient Boosted Trees model, a Support Vector Machine, or a shallow neural network.

8. The method of claim 1, wherein parameters of the machine learning algorithm are periodically updated by incremental learning that appends newly acquired status sheets and outcome events to the training dataset.

9. The method of claim 1, wherein the training the model of the machine learning algorithm comprises generating a temporal feature vector derived from the plurality of dated status sheets, the temporal feature vector encoding changes in said at least four binary observational indicators over at least two consecutive status sheets, including smoothed trend attributes.

10. The method of claim 1, wherein the step of applying the trained model to a new status sheet comprises generating a temporal difference vector between the new status sheet and at least one previously recorded status sheet; and computing the numerical risk score based on a combination of the temporal difference vector and stored parameters of the trained model.

11. A computer-implemented method executed by a processor for assessing, within a predetermined future time window, a risk of hospital transfer of a monitored individual,
the method comprising:
a data collection phase, comprising:
acquiring, for a group of individuals, a plurality of dated status sheets, each dated status sheet comprising:
a plurality of binary observational indicators, each binary observational indicator relating to at least one of: health, social interaction, behavior, and physical or sensory capabilities of the individual; and
at least one physiological parameter selected from blood pressure, heart rate, body temperature, oxygen saturation, and body weight; and
acquiring outcome data relating to hospital admission events for at least some individuals of the group;
a model training phase, comprising:
building a training dataset combining: the plurality of dated status sheets, the outcome data, and a set of negative status sheets from individuals without imminent transfer; and
training, by the processor, a hybrid model of a machine learning algorithm comprising at least a classifier that integrates the plurality of binary observational indicators as primary features and said at least one physiological parameter as a secondary feature, on the training dataset to provide a trained hybrid model; and
an operational analysis phase, comprising:
acquiring, for the monitored individual, a plurality of new status sheets established at distinct time points;
extracting, for each new status sheet, temporal attributes representing changes in the plurality binary observational indicators as well as smoothed trends of said at least one physiological parameter;
applying the trained hybrid model to compute a numerical risk score, wherein the contribution of said plurality of binary observational indicators is weighted higher than that of said at least one physiological parameter;
updating the numerical risk score at predefined analysis intervals based on at least a new status sheet;
automatically generating an alert when the numerical risk score or a temporal trend of the numerical risk score exceeds a threshold and transmitting the alert to a monitoring platform or healthcare professional.

12. The method of claim 11, wherein the plurality of binary observational indicators is selected among:
indicators related to a health condition of the individual, comprising:
A1. the individual has swollen legs;
A2. the individual has difficulties in breathing;
A3. the individual is feverish; and
A4. the individual has pains;
relational-type indicators, comprising:
B1. the individual is indifferent;
B2. the individual is not very communicative;
B3. the individual lives alone since at least seven days; and
B4. the individual has contacts or visits with his entourage;
behavioral-type indicators, comprising:
C1. the individual refuses help with toileting;
C2. the individual does not recognize the companion;
C3. the individual forgets when the companion has come by;
C4. the individual communicates inconsistently;
C5. the individual is aggressive;
C6. the individual is sad;
C7. the individual stores objects in inappropriate locations;
C8. the individual seems tired;
C9. the individual refuses the intervention of the companion; and
C10. the individual gets dressed; and
indicators representative of the physical or sensory capabilities of the individual, comprising:
D1. the individual stands up;
D2. the individual moves at his home;
D3. the individual performs personal hygiene;
D4. the individual prepares his meals;
D5. the individual leaves his home;
D6. the individual eats; and
D7. the individual falls.

13. The method of claim 11, wherein the hybrid model assigns a weighting coefficient to each binary observational indicator that is greater than a weighting coefficient assigned to each physiological parameter.

14. A computer-implemented method executed by a processor for predicting, within a predetermined future time window, an onset of at least one symptom in a monitored individual in an everyday environment, the method comprising:
a data collection phase, comprising:
acquiring, for a group of individuals, a plurality of dated status sheets, each dated status sheet comprising at least four binary observational indicators relating to at least one of: health, social interaction, behavior, and physical or sensory capabilities of the individual, without any physiological parameter; and
acquiring outcome data relating to occurrences of symptoms for at least some individuals of the group; and
a model training phase, comprising:
building an initial training dataset combining: the plurality of dated status sheets, the outcome data, and a set of negative status sheets from individuals without imminent transfer;
training, by the processor, a model of a machine learning algorithm on the training dataset to provide a trained model;
an operational analysis phase, comprising:
acquiring, for the monitored individual, a plurality of new status sheets established at distinct time points;
applying the trained model to compute a numerical risk score for said at least one symptom, said at least one symptom not having been previously observed for the monitored individual in recorded status sheets;
updating the numerical risk score at predefined analysis intervals based on at least a new status sheet;
automatically generating an alert when the numerical risk score or a temporal trend of the numerical risk score exceeds a threshold and transmitting the alert to a monitoring platform or healthcare professional.

15. The method of claim 14, wherein said at least one symptom predicted is selected from a risk of falling, a risk of malnutrition, a risk of depression, or a risk of swollen legs.

16. The method of claim 14, wherein the trained model produces an explainability output comprising at least one feature-importance score for indicators that contributed to a prediction of said at least one symptom.

17. A computer implemented system to remotely monitor and assess, within a predetermined future time window, a health risk in a monitored individual, the system comprising:
a processor; and
a memory to store instructions which, when executed by the processor, cause the processor to:
acquire, for a group of individuals, a plurality of dated status sheets, each dated status sheet comprising at least four binary observational indicators, each binary observational indicator relating to at least one of: health, social interaction, behavior, and physical or sensory capabilities of the individual, without any physiological parameter;
acquire outcome data relating to hospital admission events for at least some individuals of the group;
build an initial training dataset combining the acquired status sheets, the outcome data, and a set of negative status sheets;
train a model of a machine learning algorithm on the training dataset to provide a trained model;
acquire a plurality of new status sheets of the monitored individual established at distinct time points;
apply the trained model to compute a numerical risk score of hospital transfer or an onset of at least one symptom in the monitored individual;
update the numerical risk score at predefined analysis intervals; and
automatically generate and transmit an alert when the numerical risk score or a temporal trend of the numerical risk score exceeds a threshold.

18. The computer system of claim 17, wherein the memory further stores instructions, when executed by the processor, cause the processor to generate a temporal feature vector encoding changes of said at least four binary observational indicators across at least two consecutive status sheets.

19. The computer system of claim 17, further comprising at least one motion detection sensor selected from a presence sensor, a camera, or an infrared camera, the sensor being configured to automatically populate at least one binary indicator of said plurality of dated status sheets.

20. A non-transitory computer-readable medium storing instructions that, when executed by a processor, cause the processor to:
acquire, for a group of individuals, a plurality of dated status sheets, each dated status sheet comprising at least four binary observational indicators, each binary observational indicator relating to at least one of: health, social interaction, behavior, and physical or sensory capabilities of the individual, without any physiological parameter;
acquire outcome data relating to hospital admission events for at least some individuals of the group;
build an initial training dataset combining the acquired status sheets, the outcome data, and a set of negative status sheets;
train a model of a machine learning algorithm on the training dataset to provide a trained model;
acquire a plurality of new status sheets of the monitored individual established at distinct time points;
apply the trained model to compute a numerical risk score of hospital transfer or an onset of at least one symptom in the monitored individual;
update the numerical risk score at predefined analysis intervals; and
automatically generate and transmit an alert when the numerical risk score or a temporal trend of the numerical risk score exceeds a threshold.
